(19) United States
(12) Patent Application Publication
(10) Pub. No.: US 2017/0079359 A1
(43) Pub. Date: Mar. 23, 2017

(54) Modular/Multifunction Hat with Removable Face Coverage

(71) Applicant: Daniel Justin Chase, Loomis, CA (US)

(72) Inventor: Daniel Justin Chase, Loomis, CA (US)

(21) Appl. No.: 14/857,730

(22) Filed: Sep. 17, 2015

(51) Int. Cl. A42B 1/06; A61F 9/02; A41D 23/00; A42B 1/04; A42B 1/18

(52) U.S. Cl. CPC A42B 1/066; A42B 1/068; A42B 1/067; A42B 1/046; A42B 1/18; A41D 23/00; A61F 9/02

(57) ABSTRACT

A multifunction hat system is disclosed, comprising at least a head covering unit, an ear covering unit that can be removably or permanently attached to the head covering unit, and a face covering unit that can be removably or permanently attached to either the head covering unit, the ear covering unit, or both. Other modules, such as a scarf, ski goggles, earphones, safety lights, a helmet, and so on, may also be removably or permanently attached.

### RELATED APPLICATIONS

This application is a continuation in part of application Ser. No. 13/926,448, filed Jun. 25, 2013, which claims priority from U.S. Provisional Application No. 61/663,795, filed on Jun. 25, 2012, which is incorporated by reference herein in its entirety.

### FIELD OF THE INVENTION

The invention relates to the field of headwear and more particularly winter and extreme weather hats that are modular/multifunctional and contain several elements that can be attached and detached or flipped in and out of place.

### BACKGROUND

Warm winter clothing, including hats, is essential for the comfortable enjoyment of winter sports and outdoor activities. If the temperature is sufficiently low, a hat that covers the ears is preferable; if it is even lower, a face mask that also covers the face is preferable. Furthermore, headwear is also useful for protection from other forms of extreme weather, such as strong wind or rain. In summer weather, hats are essential for protecting the wearer against the sun or wind, and a face mask is useful for protection against pollution, car exhaust, or other similar air contaminants.

There are many winter hats on the market that include ear protection, and balaclavas or face masks that are separate; however, those hats can be overly warm when the temperature rises and none of them are modular/multifunctional. Depending on the activity and the weather, a user may require a full face mask in the morning, and only a simple hat in the afternoon. This means that for comfortable enjoyment of an extreme-weather activity, a user may have to carry several hats of different level of warmth. While it is theoretically possible to carry a separate face mask, the gaps between the face mask and the hat may let in cold air and snow and make it uncomfortable for the user. In the summer, a user may require a face mask only some of the time, and may want their face free some other times.

Furthermore, a user may need a hat to which other modules may be attached—for example, ski goggles, a helmet shell, headphones, safety lights, or the microphone for a walkie-talkie. For travel or extreme sports, other modules may be required—a virtual-reality goggle such as the Google Glass, pockets, an inflatable pillow, a USB port, and so on. In the summer, a cooling module (whether a fan or a special fabric that can be wetted) may be necessary to prevent heatstroke. A special wicking fabric may also be used to minimize sweating and to keep the user dry. This would be beneficial in cold or warm weather.

A need therefore exists for a multifunction hat system that can be changed from a simple hat that protects the head, to a hat that also protects the ears, to a hat with a face mask that also protects the face, to a hat with a scarf that protects the neck, and so on, depending on the modules desired.

### SUMMARY OF THE INVENTION

An object of the present invention is to provide a modular hat that may be customized to meet the needs of the user at a given time and in given weather conditions.

Another object of the present invention is to provide a modular hat comprising a head covering unit, at least one ear covering unit that covers at least one of the user's ears, and a face covering unit, all of which are attached to each other. The ear covering unit preferably covers both ears and the back of the user's neck, and attaches to the head covering unit by a permanent or removable fastener that extends over the entire length of the ear covering unit and part of the circumference of the head covering unit.

In an alternate embodiment of the invention, the face covering unit comprises a left half and a right half, the two halves joined together by a fastener at the front or slightly to the side of the user's face. When the face covering unit is not needed, it may be stowed away at the back of the user's head by unfastening the fastener, folding up each half individually, and fastening them to the back of the head covering unit in a folded position by means of a fastener such as a Velcro strap or a strap with a snap, or by inserting them into a pocket.

In one embodiment of the invention, the modular hat also comprises a scarf removably or permanently attached to the head covering unit, the ear covering unit, or the face covering unit. The attachment means may be a sewn connection, snaps, buttons, zippers, Velcro, or any other fastener capable of securely attaching the scarf.

In one embodiment of the invention, the modular hat also comprises ski goggles removably or permanently attached to the head covering unit, the face covering unit, or both. The attachment means may be a sewn connection, snaps, zippers, buttons, Velcro, or any other fastener capable of securely attaching the ski goggles.

In one embodiment of the invention, the modular hat also comprises a removable air filter. The air filter can be an activated carbon filter, a woven or nonwoven filter, a foam, an activated charcoal cloth, or a respirator such as an air purifying escape respirator or a positive pressure respirator.

In one embodiment of the invention, the face covering unit also comprises a moldable wire in its top edge that can be used to mold the face covering unit to the user's facial contours and prevent cold air from getting in.

In one embodiment of the invention, the modular hat also comprises a visor removably or permanently attached to the head covering unit. The attachment means may be a sewn connection, snaps, zippers, buttons, Velcro, or any other fastener capable of securely attaching the visor.

In one embodiment of the invention, the modular hat also comprises a helmet shell removably or permanently attached to the head covering unit and possibly also to the ear covering unit. The helmet shell may attach on the inside or the outside of the head covering unit and ear covering unit, or may be permanently attached between two layers of fabric. The attachment means may be adhesives, sewing, snaps, zippers, buttons, Velcro, or any other fastener capable of securely attaching the helmet shell.

In other embodiments of the invention, the modular hat may also comprise various communication, safety, and/or entertainment modules, such as headphones, Bluetooth headsets, walkie-talkie microphones, GPS modules, safety lights, and so on. These modules are also removably or permanently attached to the multifunction hat by fasteners such as sewing, snaps, zippers, buttons, Velcro, or any other fasteners suitable for the purpose.

### BRIEF DESCRIPTION OF THE SEVERAL VIEWS OF THE DRAWINGS

FIG. 1 is a front view of the preferred embodiment of the present invention.

FIG. 2 is a side view of the preferred embodiment of the present invention.

FIG. 3A is a front view of an alternate embodiment of the present invention, showing a face mask that zips together in the front.

FIG. 3B is a back view of the same embodiment shown in FIG. 3A, showing the face mask zipped together in the back.

FIG. 4 is a view of another alternate embodiment of the present invention, showing ski goggles.

FIG. 5 is a view of the mask unit of the present invention, showing an air filter and a shaping wire.

FIG. 6 is a view of an alternate embodiment of the present invention, showing a pocket, an air filter, goggles, an internal helmet, and a shaping wire.

### DETAILED DESCRIPTION OF THE PREFERRED EMBODIMENT

A preferred embodiment of the present invention is now described. Reference in the specification to “one embodiment” or to “an embodiment” means that a particular feature, structure, or characteristic described in connection with the embodiments is included in at least one embodiment of the invention. The appearances of the phrase “in one embodiment” or “an embodiment” in various places in the specification are not necessarily all referring to the same embodiment.

The language used in the specification has been principally selected for readability and instructional purposes, and may not have been selected to delineate or circumscribe the inventive subject matter. Accordingly, the disclosure of the present invention is intended to be illustrative, but not limiting, of the scope of the invention.

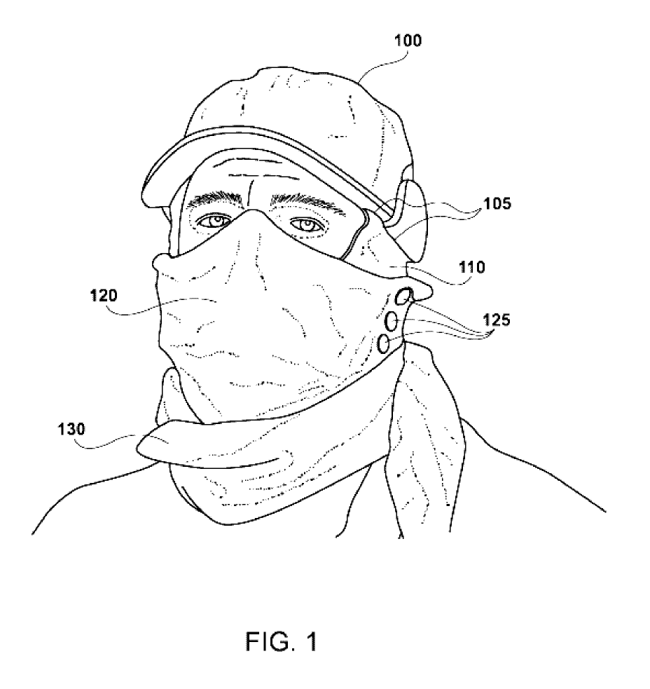

As shown in FIG. 1, the invention is a hat made for cold or extreme weather that is modular, preferably comprising a head covering unit 100, an ear covering unit 110, a mask unit 120, and, in the preferred embodiment, a detachable scarf unit 130 as well. The head covering unit 100 can be shaped like a “beanie”, a cap with a visor, or any other hat style that covers the head. The ear covering unit 110 attaches to the head covering unit 100 in a removable or permanent manner, preferably by zipper 105, and is preferably shaped in such a way as to cover both ears and the back of the neck. The ear covering unit 110 may also comprise pockets for headphones. The mask unit 120 attaches to the ear covering unit 110 in a removable or permanent manner by means of fasteners 125, and is preferably shaped in such a way as to cover the user's nose, mouth, and cheeks. (In an embodiment, the mask unit may be attached to the head covering unit 100 instead) The mask unit 120 may also comprise breathing holes to reduce condensation, or may comprise an air filter to improve breathing in poor air quality locations. The mask unit is preferably flexible in positioning so as to provide comfort regardless of climate conditions.

Scarf unit 130 is preferably attached to the back of the ear covering unit 110 by fasteners. The fasteners may be snaps, buttons, Velcro, or any other permanent or removable fastener. The scarf unit 130 may also be attached to the back of the head covering unit 100 or to the mask unit 120 instead of the ear covering unit 110.

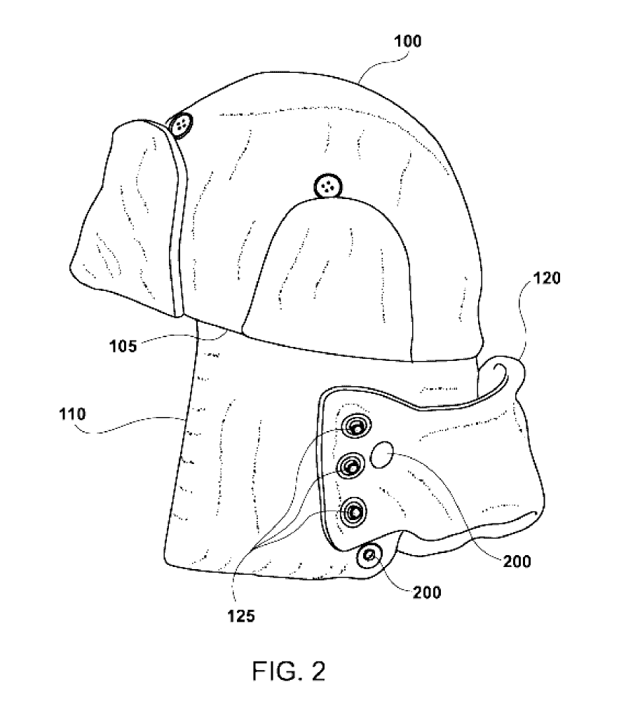

The mask unit 120 is preferably attached to the back of the head covering unit 100 or the ear covering unit 110 by fasteners 125, as shown in FIG. 2. The fasteners 125 may be snaps, buttons, Velcro, or any other removable fastener. In the preferred embodiment, fasteners 125 are snaps. In an embodiment, other fasteners may be present to secure the mask unit to the head covering unit when it is not in use, in such a way as to leave the user's face free, so as to keep it with the head covering unit and prevent it from being lost.

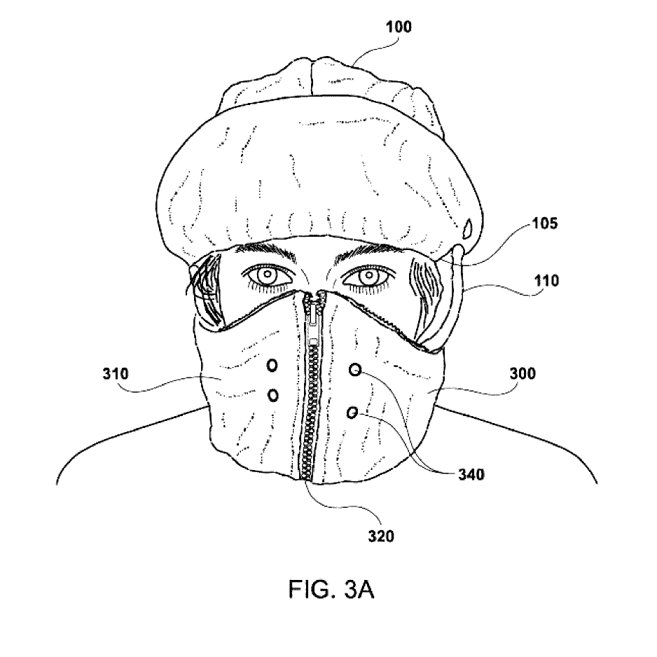

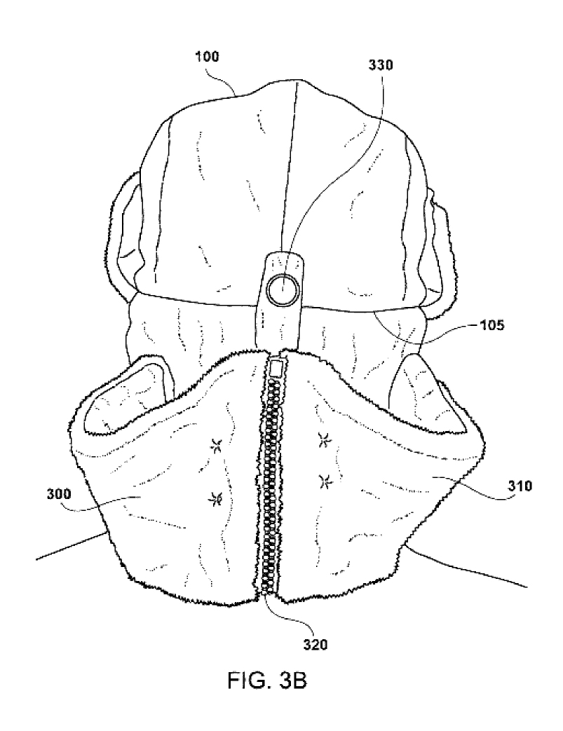

In an alternate embodiment shown in FIG. 3A, the mask unit comprises a left half 300 and a right half 310. Ventilation holes 340 are placed in front of the user's nose and/or mouth to improve ventilation. The left half 300 and right half 310 connect together in front of the user's face by a zipper 320, and are attached to the back of the head covering unit by fasteners 125. In an embodiment, the left half and right half are not the same in size and the fasteners are located off to the side of the user's face rather than in the middle. When not in use, the user can entirely detach the mask unit by detaching the fasteners 125 at the back of the head covering unit 100 and the ear covering unit 110, or unzip the zipper 320 connecting the left half 300 and right half 310 and stow away the left half 300 and right half 310 by folding them and fastening them to the back of the head covering unit by fasteners 330, as shown in FIG. 3B.

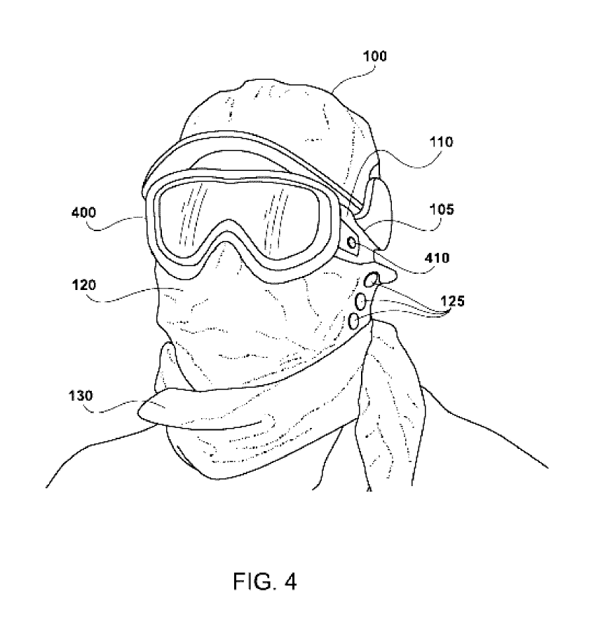

In an alternate embodiment shown in FIG. 4, ski goggles 400 may be attached to the head covering unit 100, and possibly also to the mask unit 120 or the ear covering unit 110, by fasteners 410. The fasteners are preferably snaps, but may also be Velcro, buttons, or any other fastener suitable for attaching ski goggles to a hat in a way that is secure and stable, or may be permanently sewn in or attached in some other permanent way. The ski goggles preferably seal all the way around the eyes to prevent cold air or snow from getting in.

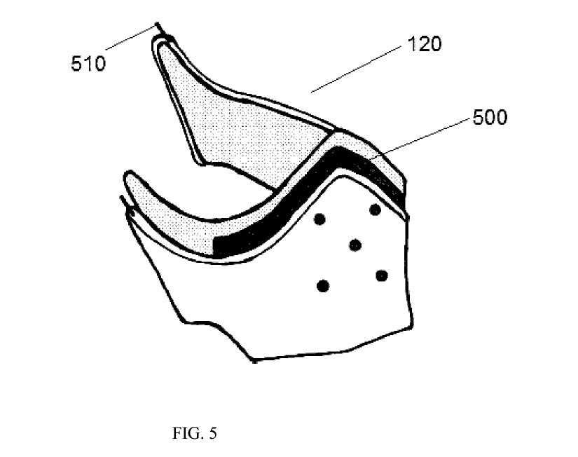

In an alternate embodiment shown in FIG. 5, the mask unit 120 may comprise a filter insert 500 capable of filtering the air breathed in by the user to remove smog particles, bacteria, pollen, dust, or other air pollutants. The filter insert 500 is preferably removable and replaceable. In the preferred embodiment, the filter insert is an activated charcoal cloth, but any air filter may be used with the present invention. Some examples are a needle punched Nomex filter, melt blown filter fabrics, various synthetic materials, foams, or powered respirators such as APER (air purifying escape respirator) or PAPR (positive air pressure respirator). In an embodiment, the air filter may comprise replaceable cartridges; alternately, the filter may be disposable and replaceable altogether. Different types and ratings of filter may be available to meet the needs of different users, or to meet an individual user's needs in different situations.

As shown in FIG. 5, the mask unit 120 may also comprise a moldable wire 510 embedded in the top edge of the mask unit. The moldable wire 510 may then be shaped by the user to conform closely to the contours of the user's face. In the preferred embodiment, the wire is made of copper and is of such a thickness as to be easily bent by a human with normal hand strength. However, the wire may be made of aluminum, moldable plastic, sculpture wire, galvanized wire, and so on. The material is required to be rigid yet flexible—i.e. a wire that can be bent into a particular shape and then holds that shape. The wire may cover the entire top edge of the mask unit or may just cover the part on and around the bridge of the nose. While in the preferred embodiment, the moldable wire is embedded in the top edge of the mask unit, multiple wires may also be used and located in other parts of the mask unit to provide extra support to the mask unit. The moldable wire may be used to flex the mask unit out of the way when not needed.

In an embodiment, the hat and mask unit may be made of breathable material stretched over the portions of the face a user wishes to cover or protect.

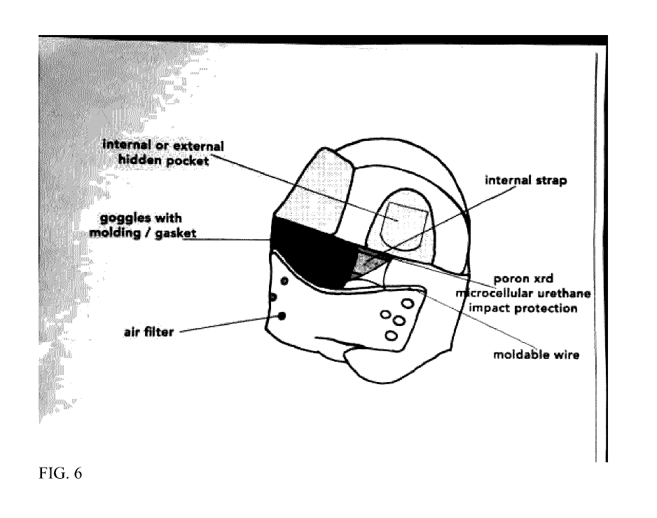

In an alternate embodiment shown in FIG. 6, the modular hat may be shaped and sized in such a way as to allow a helmet insert to be placed inside the hat, so that the user's head can be protected during more dangerous winter sports. The helmet insert is placed inside the head covering unit and the ear covering unit, and fastened to both of them by fasteners. In an alternate embodiment, the helmet insert may be placed on the outside of the head covering unit and the ear covering unit and fastened to both of them by fasteners. In that embodiment, the helmet insert may comprise fasteners suitable for attaching the mask unit, and fasteners suitable for stowing away mask unit when it is not needed. In the preferred embodiment, the helmet insert is actually inserted permanently between two layers of fabric. The helmet insert is preferably made of Poron XRD multicellular urethane, but may be made of any other material that is capable of similar levels of impact protection.

In an alternate embodiment (not shown), the mask unit may also comprise a rigid shell similar in rigidity and material to the helmet insert. The rigid shell may be connected to helmet insert by hinges that enable it to swing up onto the top of the user's head or down below the user's chin when not needed.

In an alternate embodiment (not shown), the ear covering unit and the face covering unit may comprise communication devices such as a microphone and earphones. The microphone and earphones may be wireless (such as Bluetooth) or may be connected to a device such as a walkie-talkie or a cell phone by wires.

In an alternate embodiment (not shown), the mask unit or the ear covering unit can attach to the collar of a jacket or sweatshirt to prevent cold air from getting through the gaps. The fasteners can be Velcro, clips, snaps, zippers, or any other fasteners known in the art.

In an alternate embodiment, as shown in FIG. 6, the present invention comprises pockets, whether internal or external. Such pockets may be located on the head covering unit, ear covering unit, mask unit, scarf, or anywhere else on the hat.

The present invention can be made of any material suitable for making headwear, such as cloth, fur, knitted fabric, artificial fur, leather, and any other material that has the flexibility, insulating properties, and softness necessary for headwear making purposes. The preferred embodiment is made of a cloth fabric lined with artificial fur. In an embodiment, the present invention is made wholly or partially of reflective fabric to improve visibility and safety.

1. An article of apparel, comprising:
a head covering unit, capable of covering at least the top of a user's head;
at least one ear covering unit, capable of covering at least one of a user's ears, attached to the head covering unit;
a face covering unit, capable of covering at least a user's nose and mouth, attached to one of the following group: the head covering unit, the ear covering unit;
a scarf, attached to one of the following group: the face covering unit, the ear covering unit.

2. The article of apparel of claim 1, wherein the face covering unit comprises a top side and a bottom side, wherein the top side of the face covering unit comprises a moldable wire whose shape may be changed to adjust to a user's facial contours.

3. The article of apparel of claim 1, wherein the face covering unit comprises a removable air filter.

4. The article of apparel of claim 1, wherein the air filter is one of the following group: an activated carbon filter, a woven filter, a nonwoven filter, a foam, an activated charcoal cloth, an air purifying escape respirator, a positive pressure respirator.

5. The article of apparel of claim 1, further comprising ski goggles, attached to one of the head covering unit, the face covering unit, or the ear covering unit, wherein the ski goggles comprise a top edge and a bottom edge, wherein the bottom edge of the ski goggles contacts the face covering unit while the top edge of the ski goggles contacts the head covering unit.

6. The article of apparel of claim 1, further comprising a helmet shell, attached to at least one of the head covering unit and the ear covering unit.

7. The article of apparel of claim 1, wherein the head covering unit comprises an inner layer of fabric and an outer layer of fabric, wherein the helmet shell is inserted between the inner layer of fabric and the outer layer of fabric.

8. The article of apparel of claim 6, wherein the helmet shell is made of one of the following: foam, microcellular urethane.

9. The article of apparel of claim 1, further comprising a communication device attached to at least one of the head covering unit, the ear covering unit, and the face covering unit.

10. The article of apparel of claim 1, where the ear covering unit comprises at least one pocket of such a size and shape as to fit an earphone, located in such a way that the earphone is located next to the user's ear.

11. The article of apparel of claim 1, where the at least one ear covering unit is attached to the head covering unit by at least one of the following group: snaps, buttons, Velcro, or a zipper.

12. The article of apparel of claim 1, where the at least one ear covering unit covers both of the user's ears and the back of the user's neck.

13. The article of apparel of claim 1, where the face covering unit is attached by at least one of the following group: snaps, buttons, Velcro, or a zipper.

14. The article of apparel of claim 1, wherein the scarf is attached by at least one of the following group: snaps, buttons, Velcro, or a zipper.

15. The article of apparel of claim 1, where the face covering unit comprises:
a left half attached to one of the following: the head covering unit, the ear covering unit, at the back of the user's head;
a right half attached to one of the following: the head covering unit, the ear covering unit, at the back of the user's head;
a fastener positioned in such a way as to connect the left half and the right half

16. The article of apparel of claim 15, where the fastener is one of the following: a zipper, Velcro, snaps, buttons.

17. The article of apparel of claim 15, further comprising fasteners placed in such a way as to secure the left half and the right half in a position that leaves the user's face uncovered.

18. The article of apparel of claim 1, where the ear covering unit comprises a top side and a bottom side, where the bottom side of the ear covering unit comprises fasteners capable of attaching the ear covering unit to a collar of a garment.
